(19) United States
(12) Patent Application Publication
(10) Pub. No.: US 2015/0050665 A1
(43) Pub. Date: Feb. 19, 2015

(54) PREDICTIVE BIOMARKERS FOR BREAST CANCER

(71) Applicant: Nuclea Biotechnologies, Inc., Pittsfield, MA (US)

(72) Inventor: Patrick J. Muraca, Pittsfield, MA (US)

(21) Appl. No.: 14/516,844

(22) Filed: Oct. 17, 2014

Related U.S. Application Data
(63) Continuation of application No. 13/651,588, filed on Oct. 15, 2012.

(51) Int. Cl. G01N 33/574

(52) U.S. Cl. CPC G01N 33/57484; G01N 33/57415; G01N 2333/91057
USPC 435/7.4

(57) ABSTRACT

The invention relates to compositions and methods for detecting, screening, diagnosing or determining the progression of, regression of and/or survival from a proliferative disease or condition, specifically breast cancer. The invention also provides new assays and kits for the staging or stratifying breast cancer patients or patients suspected of having breast cancer.

### CROSS REFERENCE TO RELATED APPLICATIONS

This application is a continuation of U.S. application Ser. No. 13/651,588, filed Oct. 15, 2012, which in turn claims priority to U.S. Provisional Patent Application No. 61/547,838 filed Oct. 17, 2011 and U.S. Provisional Patent Application No. 61/605,798 filed Mar. 2, 2012, each of which is incorporated by reference in its entirety.

### REFERENCE TO SEQUENCE LISTING

The present application is being filed along with a Sequence Listing in electronic format. The Sequence Listing is provided as a file entitled 20151006USSEQLST.txt, created on Oct. 15, 2012, which is 39.6 KB (40,621 bytes) in size. The information in the electronic format of the sequence listing is incorporated herein by reference in its entirety.

### FIELD OF THE INVENTION

The invention relates to compositions, methods, assays and kits for detecting, screening, diagnosing or determining the progression of, regression of and/or survival from a proliferative disease or condition, in particular breast cancer.

### BACKGROUND OF THE INVENTION

Breast cancer is the most common form of cancer in women and is second only to lung cancer as a cause of death. It is estimated that, based on current incidence rates, an American women has a one in eight chance of developing breast cancer at some time during her life. According to the American Cancer Society, in 2010, an estimated 207,090 new cases of invasive breast cancer were expected to be diagnosed in women in the U.S., along with 54,010 new cases of non-invasive (in situ) breast cancer.

As would be expected for such a major disease, the first efforts to apply emerging molecular and immunohistochemistry techniques in the 1980s to human cancers focused on breast cancer. Initial work considered the amplification of dormant oncogenes as prognostic markers and subsequently featured assessment of tumor suppressor genes. This was accompanied by a great interest in invasion and metastasis markers initially evaluated by immunohistochemistry and subsequently studied by molecular biologic techniques.

The morphologic features of primary breast cancer specimens and the pathologic stages that are determined from local and more extensive specimen resections have been the fundamental predictors of prognosis in the disease for more than a century. The morphologic prognosis parameters or features remain the cornerstone of predicting disease outcome. Current general consensus holds that axillary lymph node metastasis is the most significant morphologic prognosis parameter or feature, followed by tumor type, tumor grade, and tumor size. Additional important morphologic parameters generally considered to be predictive are the extent of an intraductal component in patients with mixed intraductal and infiltrating ductal carcinoma, proven intralymphatic and intravascular invasion, and high mitotic index. Ninety percent of breast cancers are ductal in origin including infiltrative and mixed infiltrative and in-situ cases. The impact of tumor type is not significant for the vast majority of patients with the disease.

Despite the predictive value of morphologic prognostic evaluation, approximately 25%-30% of patients with lymph node negative breast cancer will relapse and die from the disease and approximately 25% of patients with lymph node positive tumors will not relapse and die of the disease.

Fatty acid synthase (FAS, FASN), a 270 kDa protein is found in tumor cells from breast carcinomas of patients whose prognosis is very poor. Although the biochemical and metabolic basis for FAS expression in tumor cells in not well understood, it appears that FAS expression confers a selective advantage compared to normal cells.

It is thought that FAS regulates progression of the cell division cycle from the G₁ into S phase by binding to and thus inhibiting the cyclin E/Cdk2 complex. It has been recently reported that reduced expression of FAS correlates with poor survival in cohorts of breast carcinoma as well as colon carcinoma patients.

Immunohistochemical analysis of FAS expression in primary breast cancers has shown that low nuclear expression of FAS protein is a significant predictor of very poor disease-free survival. Results of a study using tumors from a selection of younger women, which were selected on the basis of a higher cancer death rate, showed that decreased levels of FAS were a significant predictor of poor overall survival by multivariate regression analysis.

The recent advances in breast cancer detection have made it possible to detect very small invasive breast carcinomas, which has resulted in an increased number of diagnosed cases of in-situ carcinoma. While there are a number of prognostic markers already in use for breast cancer to date, the need for a powerful prognostic marker such as describe herein is apparent.

### SUMMARY OF THE INVENTION

The present invention provides methods, assays and kits for the prediction of clinical outcomes for patients with early stage (node negative) breast cancer. These include assays which involve immunohistochemical techniques and may involve the use of FAS antibodies which may be labeled with a detectable label.

In one embodiment is provided a method of predicting a clinical outcome of a patient diagnosed with breast cancer, the method comprising obtaining a tissue sample from the patient by excision, aspiration or biopsy, assaying the sample by one or more colorimetric methods to determine the nuclear stain intensity and stain positivity of fatty acid synthase, and classifying as a good clinical outcome any assay results where stain intensity and stain positivity are individually at least 2.00.

Stain intensities and stain positivities may vary within certain ranges. These ranges for stain intensity include from about 2.45 to about 4. In this range the stain positivity may be from about 3.69 to about 4. Further ranges for stain intensity include from about 2.69 to about 4. In this range the stain positivity may be from about 3.23 to about 4. Further ranges for stain intensity include from about 3.10 to about 4. In this range the stain positivity may be from about 3.00 to about 4. Further ranges for stain intensity include from about 3.23 to about 4. In this range the stain positivity may be from about 2.55 to about 4. Further ranges for stain intensity include from about 3.69 to about 4. In this range the stain positivity may be from about 2.45 to about 4.

In one embodiment the sample from the patient is selected from the group consisting of epithelial cells or tissue, ductal components, lymph fluid and inflammatory cells or combinations of these. Patients may have been previously diagnosed with cancer which is lymph node negative or positive.

Assays of the present invention include immunoassays. These may include any immunoassay including but not limited to ELISAs, IHC or other colorimetric assay.

The antibodies used may be polyclonal or monoclonal antibodies and may contain a detectable label.

In one embodiment, a method for predicting the of likelihood of a metastatic event occurring within five years in a lymph node negative patient diagnosed with breast cancer independent of age, Her2/neu status or estrogen receptor status is provided comprising obtaining a breast tissue sample from the patient by excision, aspiration or biopsy, assaying the sample to determine the nuclear stain positivity boundary pair scores of fatty acid synthase, classifying the results as likely to predict the occurrence of a metastatic event in the patient when the boundary pair scores are individually less than 2.00.

### DETAILED DESCRIPTION OF THE INVENTION

The present invention provides a method for the stratification of patients with breast cancer. The invention provides a superior assay, involving the measurement and/or scoring of FAS expression, for the prediction of outcomes for patients with early stage (node negative; T1NOMO, where (T1) clinical stage I; (NO) Node Negative; and (MO) is No Mets) breast carcinoma.

The methods provided here represent an improvement over mere morphologic parameter or feature assessments and those of mammography. Mammographic sensitivity ranges from 83% to 95%, and specificity ranges from 93% to 99%. Furthermore, sensitivity and specificity are lower in women who are younger than 50. Thus, 5% to 10% of all screening mammograms are reported as abnormal, and—more importantly—about 90% of women with abnormal mammograms do not have breast cancer. Appropriate and timely follow-up of abnormal mammograms is crucial for relieving patients' anxiety and for assuring prompt intervention if malignancy is present. Optimal strategies for managing patients with abnormal mammograms should allow clinicians to rapidly identify those patients who have breast cancer, and, with the same speed and accuracy, to identify (and reassure) those who do not. These strategies may now include the methods of the present invention.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below.

For convenience, the meaning of certain terms and phrases employed in the specification, examples, and appended claims are provided below. The definitions are not meant to be limiting in nature and serve to provide a clearer understanding of certain aspects of the present invention.

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The phrase “single-gene marker” or “single gene marker” refers to a single gene (including all variants of the gene) expressed by a particular cell or tissue type wherein presence of the gene or transcriptional products thereof, taken individually the differential expression of such, is indicative/predictive of a certain condition.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

The term “complementary” as it relates to nucleic acids refers to hybridization or base pairing between nucleotides or nucleic acids, such as, for example, between the two strands of a double-stranded DNA molecule or between an oligonucleotide probe and a target are complementary.

As used herein, an “expression product” is a biomolecule, such as a protein or mRNA, which is produced when a gene in an organism is expressed. An expression product may comprise post-translational modifications. The polypeptide of a gene may be encoded by a full length coding sequence or by any portion of the coding sequence.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The present invention contemplates several types of compositions, such as antibodies, which are amino acid based including variants and derivatives. These include substitutional, insertional, deletion and covalent variants and derivatives. As such, included within the scope of this invention are polypeptide based molecules containing substitutions, insertions and/or additions, deletions and covalently modifications. For example, sequence tags or amino acids, such as one or more lysines, can be added to the polypeptide sequences of the invention (e.g., at the N-terminal or C-terminal ends). Sequence tags can be used for polypeptide purification or localization. Lysines can be used to increase solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants,” when referring to proteins, are those with one or more amino acids in the native or starting amino acid sequence removed. Ordinarily, deletional variants will have one or more amino acids deleted in a particular region of the molecule.

“Covalent derivatives,” when referring to proteins, include modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol. Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

The term “protein expression” refers to the process by which a nucleic acid sequence undergoes translation such that detectable levels of the amino acid sequence or protein are expressed.

The phrase “single-protein marker” or “single protein marker” refers to a single protein (including all variants of the protein) expressed by a particular cell or tissue type wherein presence of the protein or translational products of the gene encoding said protein, taken individually the differential expression of such, is indicative/predictive of a certain condition.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

The terms “array” and “microarray” refer to any type of regular arrangement of objects usually in rows and columns. As it relates to the study of gene and/or protein expression, arrays refer to an arrangement of probes (often oligonucleotide or protein based) or capture agents anchored to a surface which are used to capture or bind to a target of interest. Targets of interest may be genes, products of gene expression, and the like. The type of probe (nucleic acid or protein) represented on the array is dependent on the intended purpose of the array (e.g., to monitor expression of human genes or proteins). The oligonucleotide- or protein-capture agents on a given array may all belong to the same type, category, or group of genes or proteins. Genes or proteins may be considered to be of the same type if they share some common characteristics such as species of origin (e.g., human, mouse, rat); disease state (e.g., cancer); structure or functions (e.g., protein kinases, tumor suppressors); or same biological process (e.g., apoptosis, signal transduction, cell cycle regulation, proliferation, differentiation). For example, one array type may be a “cancer array” in which each of the array oligonucleotide- or protein-capture agents correspond to a gene or protein associated with a cancer. An “epithelial array” may be an array of oligonucleotide- or protein-capture agents corresponding to unique epithelial genes or proteins. Similarly, a “cell cycle array” may be an array type in which the oligonucleotide- or protein-capture agents correspond to unique genes or proteins associated with the cell cycle.

The terms “immunohistochemical” or as abbreviated “IHC” as used herein refer to the process of detecting antigens (e.g., proteins) in a biologic sample by exploiting the binding properties of antibodies to antigens in said biologic sample.

The term “immunoassay” refers to a test that uses the binding of antibodies to antigens to identify and measure certain substances. Immunoassays often are used to diagnose disease, and test results can provide information about a disease that may help in planning treatment. An immunoassay takes advantage of the specific binding of an antibody to its antigen. Monoclonal antibodies are often used as they usually bind only to one site of a particular molecule, and therefore provide a more specific and accurate test, which is less easily confused by the presence of other molecules. The antibodies used must have a high affinity for the antigen of interest, because a very high proportion of the antigen must bind to the antibody in order to ensure that the assay has adequate sensitivity.

The term “PCR” or “RT-PCR”, abbreviations for polymerase chain reaction technologies, as used here refer to techniques for the detection or determination of nucleic acid levels, whether synthetic or expressed.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

The term “complementary” as it relates to arrays refers to the topological compatibility or matching together of the interacting surfaces of a probe molecule and its target. The target and its probe can be described as complementary, and furthermore, the contact surface characteristics are complementary to each other.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE, etc.

The term “antibody fragment” refers to any derivative or portion of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab)₂, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. This type of antibodies is produced by the daughter cells of a single antibody-producing hybridoma. A monoclonal antibody typically displays a single binding affinity for any epitope with which it immunoreacts.

The modifier “monoclonal” indicates the character of the antibody as being obtained from a substantially homogeneous population of antibodies, and is not to be construed as requiring production of the antibody by any particular method. Monoclonal antibodies recognize only one type of antigen The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

A monoclonal antibody may contain an antibody molecule having a plurality of antibody combining sites, each immunospecific for a different epitope, e.g., a bispecific monoclonal antibody. Monoclonal antibodies may be obtained by methods known to those skilled in the art. Kohler and Milstein (1975), Nature, 256:495-497; U.S. Pat. No. 4,376,110; Ausubel et al. (1987, 1992), eds., Current Protocols in Molecular Biology, Greene Publishing Assoc. and Wiley Interscience, N.Y.; Harlow and Lane (1988), Antibodies: A Laboratory Manual, Cold Spring Harbor Laboratory; Colligan et al. (1992, 1993), eds., Current Protocols in Immunology, Greene Publishing Assoc. and Wiley Interscience, N.Y.; Iyer et al., Ind. J. Med. Res., (2000), 123:561-564.

An “antibody preparation” is meant to embrace any composition in which an antibody may be present, e.g., a serum (antiserum).

Antibodies may be labeled with detectable labels by one of skill in the art. The label can be a radioisotope, fluorescent compound, chemiluminescent compound, quantum dot, enzyme, or enzyme co-factor, or any other labels known in the art. In some aspects, the antibody that binds to an entity one wishes to measure (the primary antibody) is not labeled, but is instead detected by binding of a labeled secondary antibody that specifically binds to the primary antibody.

Antibodies of the invention include, but are not limited to, polyclonal, monoclonal, multispecific, human, humanized or chimeric antibodies, single chain antibodies, Fab fragments, F(ab) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. The antibodies of the invention can be from any animal origin including birds and mammals. Preferably, the antibodies are of human, murine (e.g., mouse and rat), donkey, sheep, rabbit, goat, guinea pig, camel, horse, or chicken origin.

Multispecific antibodies can be specific for different epitopes of a peptide of the present invention, or can be specific for both a peptide of the present invention, and a heterologous epitope, such as a heterologous peptide or solid support material. See, e.g., WO 93/17715; WO 92/08802; WO 91/00360; WO 92/05793; Tutt et al., 1991, J. Immunol., 147:60-69; U.S. Pat. Nos. 4,474,893; 4,714,681; 4,925,648; 5,573,920; 5,601,819; and Kostelny et al., 1992, J. Immunol., 148:1547-1553. For example, the antibodies may be produced against a peptide containing repeated units of a FAS peptide sequence of the invention, or they may be produced against a peptide containing two or more FAS peptide sequences of the invention, or the combination thereof.

Moreover, antibodies can also be prepared from any region of the FAS peptides of the invention. In addition, if a polypeptide is a receptor protein, antibodies can be developed against an entire receptor or portions of the receptor, for example, an intracellular domain, an extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example.

By “amplification” is meant production of multiple copies of a target nucleic acid that contains at least a portion of an intended specific target nucleic acid sequence. The multiple copies may be referred to as amplicons or amplification products. Preferably, the amplified target contains less than the complete target gene sequence (introns and exons) or an expressed target gene sequence (spliced transcript of exons and flanking untranslated sequences). For example, FAS-specific amplicons may be produced by amplifying a portion of the FAS target polynucleotide by using amplification primers which hybridize to, and initiate polymerization from, internal positions of the FAS target polynucleotide. Preferably, the amplified portion contains a detectable target sequence which may be detected using any of a variety of well known methods.

By “primer” or “amplification primer” is meant an oligonucleotide capable of binding to a region of a target nucleic acid or its complement and promoting nucleic acid amplification of the target nucleic acid. In most cases a primer will have a free 3′ end which can be extended by a nucleic acid polymerase. All amplification primers include a base sequence capable of hybridizing via complementary base interactions either directly with at least one strand of the target nucleic acid or with a strand that is complementary to the target sequence. Amplification primers serve as substrates for enzymatic activity that produces a longer nucleic acid product.

A “target-binding sequence” of an amplification primer is the portion that determines target specificity because that portion is capable of annealing to a target nucleic acid strand or its complementary strand. The complementary target sequence to which the target-binding sequence hybridizes is referred to as a primer-binding sequence.

By “detecting” an amplification product is meant any of a variety of methods for determining the presence of an amplified nucleic acid, such as, for example, hybridizing a labeled probe to a portion of the amplified product. A labeled probe is an oligonucleotide that specifically binds to another sequence and contains a detectable group which may be, for example, a fluorescent moiety, a chemiluminescent moiety, a radioisotope, biotin, avidin, enzyme, enzyme substrate, or other reactive group.

By “nucleic acid amplification conditions” is meant environmental conditions including salt concentration, temperature, the presence or absence of temperature cycling, the presence of a nucleic acid polymerase, nucleoside triphosphates, and cofactors which are sufficient to permit the production of multiple copies of a target nucleic acid or its complementary strand using a nucleic acid amplification method. Many well-known methods of nucleic acid amplification require thermocycling to alternately denature double-stranded nucleic acids and hybridize primers.

The term “biomarker” as used herein refers to a characteristic that is objectively measured and evaluated as an indicator of normal biological processes, pathogenic processes or biological responses to a therapeutic intervention. They can suggest etiology of, susceptibility to, activity of or progress of a disease substance indicative of a biological state.

The term “biological sample” or “biologic sample” refers to a sample obtained from an organism (e.g., a human patient) or from components (e.g., cells) or from body fluids (e.g., blood, serum, sputum, urine, etc) of an organism. The sample may be of any biological tissue, organ, organ system or fluid. The sample may be a “clinical sample” which is a sample derived from a patient. Such samples include, but are not limited to, sputum, blood, blood cells (e.g., white cells), amniotic fluid, plasma, semen, bone marrow, and tissue or core, fine or punch needle biopsy samples, aspirations, urine, peritoneal fluid, and pleural fluid, or cells therefrom. Biological samples may also include sections of tissues such as frozen sections taken for histological purposes. A biological sample may also be referred to as a “patient sample.”

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

The term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

The term “breast cancer” means a cancer of the breast tissue or associated lymph nodes.

The term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

The term “tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

The term “lesion” or “lesion site” as used herein refers to any abnormal, generally localized, structural change in a bodily part or tissue. Calcifications or fibrocystic features are examples of lesions of the present invention.

The term “clinical management parameter” refers to a metric or variable considered important in the detecting, screening, diagnosing, staging or stratifying patients, or determining the progression of, regression of and/or survival from a disease or condition. Examples of such clinical management parameters include, but are not limited to survival in years, disease related death, early or late recurrence, degree of regression, metastasis, responsiveness to treatment, effectiveness of treatment or the likelihood of progression to breast cancer.

The term “endpoint” means a final stage or occurrence along a path or progression.

The term “tumor assessment endpoint” means an endpoint observation or calculation based on the stage, status or occurrence of a tumor. Examples of endpoints based on tumor assessments include, but are not limited to, survival, disease free survival (DFS), objective response rate (ORR), time to progression (TTP), progression free survival (PFS), and time to treatment failure (TTF).

The phrase “morphologic prognosis parameter or feature” means a feature of the cancerous phenotype used to predict an outcome. Morphologic prognosis parameters or features include axillary lymph node metastasis (which is the most significant), tumor type, tumor grade, and tumor size. Secondary but important morphologic parameters also considered predictive include the extent of an intraductal component in patients with mixed intraductal and infiltrating ductal carcinoma, proven intralymphatic and intravascular invasion, and high mitotic index.

The phrase “lymph node negative” as used herein refers to the status of a patient where at least one or more removed or biopsied lymph nodes showed no evidence of metastatic carcinoma. In one embodiment, a lymph node negative status is defined as the situation where more than 4, more than 5 or more than 6 removed or biopsied lymph nodes showed no evidence of metastatic carcinoma.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient with cancer. The term “treatment” as used herein, unless otherwise indicated, refers to the act of treating.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

The term “predicting” means a statement or claim that a particular event will, or is very likely to, occur in the future.

The term “prognosing” means a statement or claim that a particular biologic event will, or is very likely to, occur in the future.

The term “progression” or “cancer progression” means the advancement or worsening of or toward a disease or condition.

The term “regression” or “degree of regression” refers to the reversal, either phenotypically or genotypically, of a cancer progression. Slowing or stopping cancer progression may be considered regression.

The term “stratifying” as it relates to patients means the parsing of patients into groups of predicted outcomes along a continuum of from a positive outcome (such as disease free) to moderate or good outcomes (such as improved quality of life or increased survival) to poor outcomes (such as terminal prognosis or death).

The term “therapeutically effective agent” means a composition that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician.

The term “correlate” or “correlation” as used herein refers to a relationship between two or more random variables or observed data values. A correlation may be statistical if, upon analysis by statistical means or tests, the relationship is found to satisfy the threshold of significance of the statistical test used

### Clinical Management Parameters

The invention relates to compositions, methods and assays for detecting, screening for, or diagnosing breast cancer; staging or stratifying breast cancer patients; and determining the progression of, regression of and/or survival from breast cancer.

In doing so, the present invention provides methods, algorithms and other clinical tools to augment traditional diagnostic, prognostic and/or therapeutic paradigms. Combination approaches using one or more biomarkers in the determination of the value of one or more clinical management parameters also are envisioned. For example, methods of this invention that measure both FAS and any one or more biomarkers can provide potentially superior results to diagnostic assays measuring just one of these biomarkers absent the measurement of FAS. FAS may even be measured as the sole marker. This dual, or multi-biomarker approach, in combination with imaging techniques provides even further superiority. Any dual, or multiple, biomarker approach (with or without companion imaging) thus reduces the number of patients that are predicted not to benefit from treatment, and thus potentially reduces the number of patients that fail to receive treatment that may extend their lives significantly.

Clinical management parameters addressed by the present invention include survival in years, disease related death, early or late recurrence, degree of regression, metastasis, responsiveness to treatment, effectiveness of treatment.

Believing that FAS expression is a superior predictor of many of the clinical management parameters important to clinicians treating patients having or suspected of having breast cancer, the present invention involves the rapid identification of FAS expression in tissue, cells and/or serum.

The method generally comprises the following steps: (a) obtaining a biological sample from a cancer patient; (b) contacting the sample with a detection agent specific for FAS; (c) detecting the presence, amount or levels of FAS in (b); and (d) correlating the presence, amount or levels of FAS (alone or in combination) with the one or more clinical management parameters in order to aid in the prevention, diagnosis or treatment of breast cancer.

The biological sample may be cells or tissue, and preferably is serum or plasma containing cells. However, the cells also may be obtained from tissue samples or cell cultures such as in ex vivo or in situ methods.

The detection agent may a nucleic acid probe specific for FAS, or an anti-FAS antibody.

### FAS Probes

The present invention provides an assay method comprising novel nucleic acid based probes useful in the detection of the FAS gene or protein in a biological sample. The sample may be breast tissue or non-breast tissue. The non-breast tissue can include, for example, blood, lymph node, breast or breast cyst, nipple aspirations, kidney, liver, lung, muscle, stomach or intestinal tissue.

The present invention also includes a method for detecting and quantifying the FAS-specific RNA species. Other embodiments of the invention include methods for detecting other biomarker species, individually or in combination with each other or FAS sequences. Moreover, detection of these markers individually and in combination, are clinically important because cancers from individual patients may express one or more of the markers, such that detecting one or more of the markers decreases the potential of false negatives during diagnosis that might otherwise result if the presence of only one marker was tested.

### In Situ Hybridization (ISH) and Fluorescence In Situ Hybridization (FISH)

The present invention provides methods of detecting target nucleic acids via in situ hybridization and fluorescent in situ hybridization using novel probes. The methods of in situ hybridization were first developed in 1969 and many improvements have been made since. The basic technique utilizes hybridization kinetics for RNA and/or DNA via hydrogen bonding. By labeling sequences of DNA or RNA of sufficient length (approximately 50-300 base pairs), selective probes can be made to detect particular sequences of DNA or RNA. The application of these probes to tissue sections allows DNA or RNA to be localized within tissue regions and cell types. Methods of probe design are known to those of skill in the art. Detection of hybridized probe and target may be performed in several ways known in the art. Most prominently is through the use of detection labels attached to the probes. Probes of the present invention may be single or double stranded and may be DNA, RNA, or mixtures of DNA and RNA. They may also constitute any nucleic acid based construct. Labels for the probes of the present invention may be radioactive or non-radioactive and the design and use of such labels is well known in the art.

### FAS Antibodies

In one embodiment, the present invention utilizes anti-FAS antibodies in an ELISA assay. The anti-FAS antibodies preferably are those disclosed in PCT Publication PCT/US2010/030545 published Oct. 14, 2010, and PCT/US2010/046773 published Mar. 17, 2011, respectively.

The antibodies used in the present invention for detection or capture of FAS are novel anti-FAS antibodies that are highly specific for human FAS.

In one embodiment, commercial antibodies for the detection of FAS are used. For IHC the antibodies which may be used are the human anti-FASN Antibody, Affinity Purified (Catalog No. A301-324A) from Bethyl Laboratories (Montgomery, Tex.) and for ELISA studies, antibodies which may be used include the Fatty Acid Synthase Antibody Pair (Catalog No. H00002194-AP11) from Novus Biologicals (Littleton, Colo.). The pair contains a Capture antibody which is rabbit affinity purified polyclonal anti-FASN (100 ug) and a Detection antibody which is mouse monoclonal anti-FASN, IgG1 Kappa (20 ug).

In one embodiment, the present antibodies are monoclonal antibodies specific for a human FAS sequence selected from SEQ ID NOs. 1-5 (Table 1). FAS peptides are derived from the protein encoded by the FAS (Fatty Acid Synthase) gene; GenBank NM_{—}004104; SEQ ID NO: 6.

In another embodiment, the present antibodies are used as capture antibodies in a sandwich ELISA assay.

TABLE 1
FAS Peptides

| Hybridoma | FAS Peptide | SEQ ID |
|---|---|---|
| A | VAQGQWEPSGXAP | 1 |
| B | PSGPAPTNXGALE | 2 |
| C | TLEQQHXVAQGQW | 3 |
| D | EVDPGSAELQKVLQGD | 4 |
| E | ELSSKADEASELAC | 5 |

### FAS Antibodies and Detection Rate

In one embodiment, the FAS antibodies disclosed herein may be used in the detection of breast cancer, either alone or in combination with measurements of other biomarkers. Measurements may be made for example, in tissue, cells, serum or plasma of patients.

### Gene Expression and Localization of Expression

In one embodiment of the invention, FAS expression is measured relative to the expression of one or more additional genes and/or at one or more different biopsy sites. Comparisons of gene expression within the cancer site as compared to expression at the margin of the cancer and at sites distal from the cancer allow conclusions to be drawn about the status of a sample and whether it will become cancerous. These conclusions then allow for improved predictions about metastasis and consequently survival. Additional patient parameters also may be combined with the gene expression data to improve the predictive power of the method.

### FAS and Degree of Regression

In one embodiment, FAS expression levels are used as a predictor of the probability of cancer regression which allows stratification between POOR and GOOD outcomes for individual patients. In this method, FAS expression is correlated with degree of regression where higher FAS expression levels are predictive of clinical outcomes. It has been determined that FAS expression level is an excellent predictor of both GOOD and POOR outcomes.

### Assays and Kits

Any of the compositions described herein may be comprised in a kit. In one embodiment, antibodies to one or more of the expression products of the FAS genes disclosed herein are included. Antibodies may be included to provide concentrations of from about 0.1 μg/mL to about 500 μg/mL, from about 0.1 μg/mL to about 50 μg/mL or from about 1 μg/mL to about 5 μg/mL or any value within the stated ranges. The kit may further include reagents or instructions for creating or synthesizing further probes, labels or capture agents. It may also include one or more buffers, such as a nuclease buffer, transcription buffer, or a hybridization buffer, compounds for preparing a DNA template, cDNA, primers, probes or label, and components for isolating any of the foregoing. Other kits of the invention may include components for making a nucleic acid or peptide array including all reagents, buffers and the like and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial or similar container. The kits of the present invention also will typically include a means for containing the detection reagents, e.g., nucleic acids or proteins or antibodies, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may be re-suspended in any suitable solvent, such as DMSO.

Kits may also include components that preserve or maintain the compositions that protect against their degradation. Such kits generally will comprise, in suitable means, distinct containers for each individual reagent or solution.

Certain assay methods of the invention comprises contacting a tissue sample from an individual with a group of antibodies specific for some or all of the genes or proteins disclosed, and determining the occurrence of up- or down-regulation of these genes or proteins in the sample. The use of TMAs allows numerous samples, including control samples, to be assayed simultaneously.

The method preferably also includes detecting and/or quantitating control or “reference proteins”. Detecting and/or quantitating the reference proteins in the samples normalizes the results and thus provides further assurance that the assay is working properly. In a currently preferred embodiment, antibodies specific for one or more of the following reference proteins are included: beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

The present invention further comprises a kit containing reagents for conducting an IHC analysis of tissue samples or cells from individuals, e.g., patients, including antibodies specific for one or more proteins and for any reference proteins. The antibodies are preferably tagged with means for detecting the binding of the antibodies to the proteins of interest, e.g., detectable labels. Preferred detectable labels include fluorescent compounds or quantum dots; however other types of detectable labels may be used. Detectable labels for antibodies are commercially available, e.g. from Ventana Medical Systems, Inc.

Immunohistochemical methods for detecting and quantitating protein expression in tissue samples are well known. Any method that permits the determination of expression of several different proteins can be used. Such methods can be efficiently carried out using automated instruments designed for immunohistochemical (IHC) analysis. Instruments for rapidly performing such assays are commercially available, e.g., from Ventana Molecular Discovery Systems or Lab Vision Corporation. Methods according to the present invention using such instruments are carried out according to the manufacturer's instructions.

Protein-specific antibodies for use in such methods or assays are readily available or can be prepared using well-established techniques. Antibodies specific for the proteins herein can be obtained, for example, from Cell Signaling Technology, Inc, Santa Cruz Biotechnology, Inc. or Abcam.

### ImmunoAssays

The present invention provides for new assays useful in the diagnosis, prognosis and prediction of breast cancer and the elucidation of clinical management parameters associated with breast cancer. The immunoassays of the present invention utilize the anti-FAS polyclonal or monoclonal antibodies described herein to specifically bind to FAS in a biological sample. Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; The Immunoassay Handbook, D. Wild (Ed.), Elsevier Ltd., 2005; and the references disclosed therein.

The preferred assay format for the present invention is the enzyme-linked immunosorbent assay (ELISA) format. ELISA is a highly sensitive technique for detecting and measuring antigens or antibodies in a solution in which the solution is run over a surface to which immobilized antibodies specific to the substance have been attached, and if the substance is present, it will bind to the antibody layer, and its presence is verified and visualized with an application of antibodies that have been tagged or labeled so as to permit detection. ELISAs combine the high specificity of antibodies with the high sensitivity of enzyme assays by using antibodies or antigens coupled to an easily assayed enzyme that possesses a high turnover number such as alkaline phosphatase (AP) or horseradish peroxidase (HRP), and are very useful tools both for determining antibody concentrations (antibody titer) in sera as well as for detecting the presence of antigen.

There are many different types of ELISAs; the most common types include “direct ELISA,” “indirect ELISA,” “sandwich ELISA” and cell-based ELISA (C-ELISA). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate typically is washed with a mild detergent solution to remove any proteins or antibodies that are not specifically bound. After the final wash step the plate is developed by adding an enzymatic substrate tagged with a detectable label to produce a visible signal, which indicates the quantity of antigen in the sample.

In a typical microtiter plate sandwich immunoassay, an antibody (“capture antibody”) is adsorbed or immobilized onto a substrate, such as a microtiter plate. Monoclonal antibodies are preferred as capture antibodies due to their greater specificity, but polyclonal antibodies also may be used. When the test sample is added to the plate, the antibody on the plate will bind the target antigen from the sample, and retain it in the plate. When a second antibody (“detection antibody”) or antibody pair is added in the next step, it also binds to the target antigen (already bound to the monoclonal antibody on the plate), thereby forming an antigen ‘sandwich’ between the two different antibodies.

This binding reaction can then be measured by radio-isotopes, as in a radio-immunoassay format (RIA); by enzymes, as in an enzyme immunoassay format (EIA or ELISA); or other detectable label, attached to the detection antibody. The label generates a color signal proportional to the amount of target antigen present in the original sample added to the plate. Depending on the immunoassay format, the degree of color can be detected and measured with the naked eye (as with a home pregnancy test), a scintillation counter (for an RIA), or with a spectrophotometric plate reader (for an EIA or ELISA).

The assay then is carried out according to the following general steps:

Step 1: Capture antibodies are adsorbed onto the well of aplastic microtiter plate (no sample added);

Step 2: A test sample (such as human serum) is added to the well of the plate, under conditions sufficient to permit binding of the target antigen to the capture antibody already bound to the plate, thereby retaining the antigen in the well;

Step 3: Binding of a detection antibody or antibody pair (with enzyme or other detectable moiety attached) to the target antigen (already bound to the capture antibody on the plate), thereby forming an antigen “sandwich” between the two different antibodies. The detectable label on the detection antibodies will generate a color signal proportional to the amount of target antigen present in the original sample added to the plate.

In an alternative embodiment, sometimes referred to as an antigen-down immunoassay, the analyte (rather than an antibody) is coated onto a substrate, such as a microtiter plate, and used to bind antibodies found in a sample. When the sample is added (such as human serum), the antigen on the plate is bound by antibodies (IgE for example) from the sample, which are then retained in the well. A species-specific antibody (anti-human IgE for example) labeled with an enzyme such as horse radish peroxidase (HRP) is added next, which, binds to the antibody bound to the antigen on the plate. The higher the signal, the more antibodies there are in the sample.

In another embodiment, an immunoassay may be structured in a competitive inhibition format. Competitive inhibition assays are often used to measure small analytes because competitive inhibition assays only require the binding of one antibody rather than two as is used in standard ELISA formats. In a sequential competitive inhibition assay, the sample and conjugated analyte are added in steps similar to a sandwich assay, while in a classic competitive inhibition assay, these reagents are incubated together at the same time.

In a typical sequential competitive inhibition assay format, a capture antibody is coated onto a substrate, such as a microtiter plate. When the sample is added, the capture antibody captures free analyte out of the sample. In the next step, a known amount of analyte labeled with a detectable label, such as an enzyme or enzyme substrate, added. The labeled analyte also attempts to bind to the capture antibody adsorbed onto the plate, however, the labeled analyte is inhibited from binding to the capture antibody by the presence of previously bound analyte from the sample. This means that the labeled analyte will not be bound by the monoclonal on the plate if the monoclonal has already bound unlabeled analyte from the sample. The amount of unlabeled analyte in the sample is inversely proportional to the signal generated by the labeled analyte. The lower the signal, the more unlabeled analyte there is in the sample. A standard curve can be constructed using serial dilutions of an unlabeled analyte standard. Subsequent sample values can then be read off the standard curve as is done in the sandwich ELISA formats. The classic competitive inhibition assay format requires the simultaneous addition of labeled (conjugated analyte) and unlabeled analyte (from the sample). Both labeled and unlabeled analyte then compete simultaneously for the binding site on the monoclonal capture antibody on the plate. Like the sequential competitive inhibition format, the colored signal is inversely proportional to the concentration of unlabeled target analyte in the sample. Detection of labeled analyte can be read on a microtiter plate reader.

In addition to microtiter plates, immunoassays are also may be configured as rapid tests, such as a home pregnancy test. Like microtiter plate assays, rapid tests use antibodies to react with antigens and can be developed as sandwich formats, competitive inhibition formats, and antigen-down formats. With a rapid test, the antibody and antigen reagents are bound to porous membranes, which react with positive samples while channeling excess fluids to a non-reactive part of the membrane. Rapid immunoassays commonly come in two configurations: a lateral flow test where the sample is simply placed in a well and the results are read immediately; and a flow through system, which requires placing the sample in a well, washing the well, and then finally adding an analyte-detectable label conjugate and the result is read after a few minutes. One sample is tested per strip or cassette. Rapid tests are faster than microtiter plate assays, require little sample processing, are often cheaper, and generate yes/no answers without using an instrument. However, rapid immunoassays are not as sensitive as plate-based immunoassays, nor can they be used to accurately quantitate an analyte.

The preferred technique for use in the present invention to detect the amount of FAS in circulating cells is the sandwich ELISA, in which highly specific monoclonal antibodies are used to detect sample antigen. The sandwich ELISA method comprises the following general steps:
1. Prepare a surface to which a known quantity of capture antibody is bound;
2. (Optionally) block any non specific binding sites on the surface;
3. Apply the antigen-containing sample to the surface;
4. Wash the surface, so that unbound antigen is removed;
5. Apply primary (detection) antibodies that bind specifically to the bound antigen;
6. Apply enzyme-linked secondary antibodies which are specific to the primary antibodies;
7. Wash the plate, so that the unbound antibody-enzyme conjugates are removed;
8. Apply a chemical which is converted by the enzyme into a detectable (e.g., color or fluorescent or electrochemical) signal; and
9. Measure the absorbance or fluorescence or electrochemical signal to determine the presence and quantity of antigen.

In an alternate embodiment, the primary antibody (step 5) is linked to an enzyme; in this embodiment, the use of a secondary antibody conjugated to an enzyme (step 6) is not necessary if the primary antibody is conjugated to an enzyme. However, use of a secondary-antibody conjugate avoids the expensive process of creating enzyme-linked antibodies for every antigen one might want to detect. By using an enzyme-linked antibody that binds the Fc region of other antibodies, this same enzyme-linked antibody can be used in a variety of situations. The major advantage of a sandwich ELISA is the ability to use crude or impure samples and still selectively bind any antigen that may be present. Without the first layer of “capture” antibody, any proteins in the sample (including serum proteins) may competitively adsorb to the plate surface, lowering the quantity of antigen immobilized.

In one embodiment of the present invention, a solid phase substrate, such as a microtiter plate or strip, is treated in order to fix or immobilize a capture antibody to the surface of the substrate. The material of the solid phase is not particularly limited as long as it is a material of a usual solid phase used in immunoassays. Examples of such material include polymer materials such as latex, rubber, polyethylene, polypropylene, polystyrene, a styrene-butadiene copolymer, polyvinyl chloride, polyvinyl acetate, polyacrylamide, polymethacrylate, a styrene-methacrylate copolymer, polyglycidyl methacrylate, an acrolein-ethyleneglycol dimethacrylate copolymer, polyvinylidene difluoride (PVDF), and silicone; agarose; gelatin; red blood cells; and inorganic materials such as silica gel, glass, inert alumina, and magnetic substances. These materials may be used singly or in combination of two or more thereof.

The form of the solid phase is not particularly limited insofar as the solid phase is in the form of a usual solid phase used in immunoassays, for example in the form of a microtiter plate, a test tube, beads, particles, and nanoparticles. The particles include magnetic particles, hydrophobic particles such as polystyrene latex, copolymer latex particles having hydrophilic groups such as an amino group and a carboxyl group on the surfaces of the particles, red blood cells and gelatin particles. The solid phase is preferably a microtiter plate or strip, such as those available from Cell Signalling Technology, Inc.

The capture antibody preferably is one or more monoclonal anti-FAS antibodies described herein that specifically bind to at least a portion of one or more of the peptide sequences of SEQ ID NO. 1-5. Where microtiter plates or strips are used, the capture antibody is immobilized within the wells. Techniques for coating and/or immobilizing proteins to solid phase substrates are known in the art, and can be achieved, for example, by a physical adsorption method, a covalent bonding method, an ionic bonding method, or a combination thereof. See, e.g., W. Luttmann et al., Immunology, Ch. 4.3.1 (pp. 92-94), Elsevier, Inc. (2006) and the references cited therein. For example, when the binding substance is avidin or streptavidin, a solid phase to which biotin was bound can be used to fix avidin or streptavidin to the solid phase. The amounts of the capture antibody, the detection antibody and the solid phase to be used can also be suitably established depending on the antigen to be measured, the antibody to be used, and the type of the solid phase or the like. Protocols for coating microtiter plates with capture antibodies, including tools and methods for calculating the quantity of capture antibody, are described for example, on the websites for Immunochemistry Technologies, LLC (Bloomington, Minn.) and Meso Scale Diagnostics, LLC (Gaithersburg, Md.).

The detection antibody can be any anti-FAS antibody. Anti-FAS antibodies are commercially available, for example, from Cell Signaling Technologies, Inc., Santa Cruz Biotechnology, EMD Biosciences, and others. The detection antibody also may be an anti-FAS antibody as disclosed herein that is specific for one or more of SEQ ID NOs. 1-5. In one embodiment, the detection antibody may be directly conjugated with a detectable label, or an enzyme. If the detection antibody is not conjugated with a detectable label or an enzyme, then a labeled secondary antibody that specifically binds to the detection antibody is included. Such detection antibody “pairs” are commercially available, for example, from Cell Signaling Technologies, Inc.

Techniques for labeling antibodies with detectable labels are well-established in the art. As used herein, the term “detectable label” refers to a composition detectable by spectroscopic, photochemical, biochemical, immunochemical, or chemical means. The detectable label can be selected, e.g., from a group consisting of radioisotopes, fluorescent compounds, chemiluminescent compounds, enzymes, and enzyme cofactors, or any other labels known in the art. See, e.g., Zola, Monoclonal Antibodies: A Manual of Techniques, pp. 147-158 (CRC Press, Inc. 1987). A detectable label can be attached to the subject antibodies and is selected so as to meet the needs of various uses of the method which are often dictated by the availability of assay equipment and compatible immunoassay procedures. Appropriate labels include, without limitation, radionuclides, enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Evidot® quantum dots supplied by Evident Technologies, Troy, N.Y., or Qdot™ nanoparticles supplied by the Quantum Dot Corporation, Palo Alto, Calif.).

Preferably, the sandwich immunoassay of the present invention comprises the step of measuring the labeled secondary antibody, which is bound to the detection antibody, after formation of the capture antibody-antigen-detection antibody complex on the solid phase. The method of measuring the labeling substance can be appropriately selected depending on the type of the labeling substance. For example, when the labeling substance is a radioisotope, a method of measuring radioactivity by using a conventionally known apparatus such as a scintillation counter can be used. When the labeling substance is a fluorescent substance, a method of measuring fluorescence by using a conventionally known apparatus such as a luminometer can be used.

When the labeling substance is an enzyme, a method of measuring luminescence or coloration by reacting an enzyme substrate with the enzyme can be used. The substrate that can be used for the enzyme includes a conventionally known luminescent substrate, calorimetric substrate, or the like. When an alkaline phosphatase is used as the enzyme, its substrate includes chemilumigenic substrates such as CDP-Star® (4-chloro-3-(methoxyspiro (1,2-dioxetane-3,2′-(5′-chloro)tricyclo[3.3.1.1.-sup.3.7]decane)-4-yl)disodium phenylphosphate) and CSPD® (3-(4-methoxyspiro(1,2-dioxetane-3,2-(5′-chloro)tricyclo[3.3.1.1.sup.3.7]-decane)-4-yl)disodium phenylphosphate) and colorimetric substrates such as p-nitrophenyl phosphate, 5-bromo-4-chloro-3-indolyl-phosphoric acid (BCIP), 4-nitro blue tetrazolium chloride (NBT), and iodonitro tetrazolium (INT). These luminescent or calorimetric substrates can be detected by a conventionally known spectrophotometer, luminometer, or the like.

In one embodiment, the detectable labels comprise quantum dots (e.g., Evidot® quantum dots supplied by Evident Technologies, Troy, N.Y., or Qdot™ nanoparticles supplied by the Quantum Dot Corporation, Palo Alto, Calif.). Techniques for labeling proteins, including antibodies, with quantum dots are known. See, e.g., Goldman et al., Phys. Stat. Sol., 229(1): 407-414 (2002); Zdobnova et al., J. Biomed. Opt., 14(2):021004 (2009); Lao et al., JACS, 128(46):14756-14757 (2006); Mattoussi et al., JACS, 122(49):12142-12150 (2000); and Mason et al., Methods in Molecular Biology: NanoBiotechnology Protocols, 303:35-50 (Springer Protocols, 2005). Quantum-dot antibody labeling kits are commercially available, e.g., from Invitrogen (Carlsbad, Calif.) and Millipore (Billerica, Mass.).

The sandwich immunoassay of the present invention may comprise one or more washing steps. By washing, the unreacted reagents can be removed. For example, when the solid phase comprises a strip of microtiter wells, a washing substance or buffer is contacted with the wells after each step. Examples of the washing substance that can be used include 2-[N-morpholino]ethanesulfonate buffer (MES), or phosphate buffered saline (PBS), etc. The pH of the buffer is preferably from about pH 6.0 to about pH 10.0. The buffer may contain a detergent or surfactant, such as Tween 20.

The sandwich immunoassay can be carried out under typical conditions for immunoassays. The typical conditions for immunoassays comprise those conditions under which the pH is about 6.0 to 10.0 and the temperature is about 30 to 45° C. The pH can be regulated with a buffer, such as phosphate buffered saline (PBS), a triethanolamine hydrochloride buffer (TEA), a Tris-HCl buffer or the like. The buffer may contain components used in usual immunoassays, such as a surfactant, a preservative and serum proteins. The time of contacting the respective components in each of the respective steps can be suitably established depending on the antigen to be measured, the antibody to be used, and the type of the solid phase or the like.

### Kits

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, and the like.

The methods provided by the present invention may also be automated in whole or in part. The invention further provides kits for performing an immunoassay using the FAS antibodies of the present invention.

All aspects of the present invention may also be practiced such that a limited number of additional genes that are co-expressed with the disclosed genes (e.g., one or more genes from the GPEPs or FAS), for example as evidenced by high Pearson correlation coefficients, are included in a prognostic or predictive tests in addition to and/or in place of disclosed genes.

The invention is further illustrated by the following non-limiting examples.

### EXAMPLES

### Example 1

### Design of the Investigation

The pre-clinical study was designed to show the diagnostic superiority of the FAS assay by predicting outcomes for patients with early stage (node negative) breast carcinoma. In particular it was designed to estimate the proportion of breast cancer cases correctly diagnosed as lymph node positive by FAS scores but earlier classified as lymph node negative by morphologic methods. The sensitivity of the assay was also determined by evaluating the proportion of cases incorrectly diagnosed by FAS scores on cases correctly classified as lymph node negative by morphologic methods.

Cases with negative lymph node biopsies with known follow-up outcomes of non-metastatic (NM or MO; GOOD outcome) or metastatic (M1 or M2; POOR outcome) were diagnosed by both morphologic methods and FAS assay. Since the population chosen was well-known with respect to outcome, the proportion of M versus NM had to be considered a design parameter.

All study cases, M and NM, were previously diagnosed by morphological methods. Since morphologic methods already missed the M cases it was assumed that the probability that an (M) case will be classified as non-morphologic on repeated morphologic diagnosis was very small.

### Example 2

### Patient Population and Specimen Selection

The study consisted of 360 cases of formalin-fixed, paraffin embedded infiltrating (invasive) ductal carcinoma (IDC) of the breast originally obtained from patients during the years 1980-1985. All patients must have had lymph node biopsies or excision featuring six or more removed lymph nodes which showed no evidence of metastatic carcinoma (poor outcome). Patients with papillary, colloid, medullary and non-infiltrating (non-invasive) carcinoma were excluded.

The population of patients represented two groups of 180 patients per population. The first population (A) consisted of a sub-population of the above patient group in which all patients were correctly classified via morphological parameters (e.g., each patient at the 5 year point is alive, without disease, as verified by patient medical records, etc). These types of patients were classified as GOOD outcome patient population. The second (B) sub-population consisted of patients that were correctly classified at the time breast cancer was diagnosed but went on to have a metastatic event within five years of the original diagnosis. This totaled 360 patients in a double-blind multi-center (3 sites, I, II and III) pre-clinical study.

Review of the surgical pathology slides and reports were performed in order to determine and record tumor sizes and grade. In addition to recording the largest tumor diameter listed in the surgical pathology report, measurement of the largest diameter of continuous tumor on the microscope slide was also provided. Tumors were graded according to the modified Scarff-Bloom-Richardson system. The modified Bloom-Richardson-Elston grading system is also called the Nottingham system. In this system the cells and tissue structure of the breast cancer are examined histopathologically to determine how aggressive the cancer is. The test comprises the observation of three features when determining a cancer's grade: (a) the frequency of cell mitosis (rate of cell division), (b) tubule formation (percentage of cancer composed of tubular structures), and (c) nuclear pleomorphism (change in cell size and uniformity). Each of these features is assigned a score ranging from 1 to 3 (1 indicating slower cell growth and 3 indicating faster cell growth). The scores of each of the cells' features are then added together for a final sum that range between 3 and 9. A tumor with a final sum of 3, 4, or 5 is considered a Grade I tumor (well-differentiated). A sum of 6 or 7 is considered a Grade II tumor (moderately-differentiated), and a sum of 8 or 9 is a Grade III tumor (poorly-differentiated). Grades were reported as Grade I (scores 3-5); Grade II (scores 6-7) and Grade III (scores 8-9).

Additional morphologic features which were prognostic recorded included primary tumor therapy type (lumpectomy verses mastectomy with or without radiation), status of the resection margin, evidence of skin, nipple or lymphatic vessel invasion, and determination of the extent of an in-situ component if present should were also included.

A representative 5 micron tissue section taken from a cellular area of infiltrating carcinoma was analyzed for loss of FAS expression or low nuclear expression of FAS. All cases were recorded as the percentage of cells that are positive or negative as a result of the scoring method of FAS. All results were reported on case report forms provided in the protocol. Each sample had the following tumor markers data available: Estrogen and Progesterone receptor, the Dako HERcept Test™ HER-2/neu analysis, cathepsin D, and in cases where available, DNA ploidy analysis (either by flow cytometry of image analysis).

All cases accrued to the study had documented clinical follow-up for a minimum of five years. For each case, follow-up at year five (5) was documented as: (a) alive, well and free of disease;

(b) alive, recurrent disease; (c) dead from disease; (d) dead, from other causes with no evidence of recurrence. For patients with recurrent disease, the method of determining recurrence (biopsy proven versus other method) and method of determining death from disease (autopsy or no autopsy) were also documented.

### Example 3

### Materials

All supplies, the FAS detection kit of the invention and all primary and secondary reagents necessary to perform the assay on the 360 breast cancer cases were provided to the investigators at two sites. They were also provided with 10 paraffin sections with a thickness of 4 microns of selected tumor rich tissue blocks featuring invasive (infiltrating) breast carcinoma.

The investigators performed the morphologic prognostic analysis on the primary tumors, confirmed the lymph node negative status, and obtained and recorded the estrogen and progesterone receptor status. The investigators also confirmed the clinical outcome analyses according to the protocol for each patient.

Control slides that were reflective of all possible positive and negative interpretations were provided.

Proficiency evaluations were also performed and included twenty (20) samples at three sites (proficiency only) where the FAS status was already determined

### Example 4

### Statistical Analyses and Outcomes

The results of the scoring of the FAS loss of expression was analyzed using multivariate analysis relative to clinical outcome and other prognostic marker analyses determined for the other cases included in the pre-clinical study. Multivariate analysis was done using both logistic regression and Cox regression (proportional hazards) model.

The goal of this statistical analysis was to assess whether FAS can predict outcome of patients with early stage breast cancer based on cases that ALL have been classified as GOOD by careful morphological methods. Cases classified as POOR by morphological methods were not included in this study.

In the population of cases available for study, approximately 70% had indeed a GOOD outcome as determined by follow-up. But 30% of cases had POOR outcome and thus were incorrectly classified by morphological methods. From the total available cases, 360 cases were randomly selected with the restriction that 180 cases had POOR outcome in follow up and 180 cases had GOOD outcome in follow up.

These data were intended to answer the following main questions: (1) Can FAS correctly classify POOR outcome cases that by morphological methods are missed; and (2) Does FAS agree on cases correctly classified as GOOD by morphological methods?

The analyses showed that FAS measurements scores correctly classified 72% of the 30% POOR outcomes that morphological methods missed (or 23% out of 30%). Similarly, FAS measurement scores correctly classified 90% of the 70% the morphological methods correctly classified as GOOD outcome (or 61% out of 70%).

The data analyzed in this report consisted of 180 cases with known Poor Outcome and 180 cases with known Good Outcome. All 360 cases were of women who had originally been classified as Stage I by a panel of physicians. Cases classified as POOR by morphological methods were not available.

The AGE of the patient was available as a potential risk factor, but one that was not at all significant in contributing to an improved prediction. For each case the only information available other than the FAS-score was whether or not Poor Outcome was found after original classification as Stage I.

Cases were classified according to FAS expression according to the scoring method shown in Tables 2 and 3. A dual classification system was used for each specimen where the nuclear stain intensity is listed first, followed by the percent positivity of the cells stained listed second.

TABLE 2
Classification according to Nuclear Stain

| Intensity of Nuclear Stain | SCORE |
|---|---|
| None | 0 |
| Light brown | 1 |
| Light-Medium brown | 2 |
| Medium-Dark brown | 3 |
| Dark brown | 4 |

TABLE 3
Classification according to Percent Positivity

| Percent Positivity of | |
|---|---|
| Nuclear Stain | SCORE |
| None | 0 |
| <13% | 1 |
| 13-35% | 2 |
| >35-75% | 3 |
| >75% | 4 |

### Example 5

### Case Classifications from Multiple Sites

The data for the first twenty cases are shown in Tables 4-6. Each case material was tested at three sites, Site I, Site II and Site III. For the present analysis the scores from the three sites were first separated into a separate staining intensity and percent positivity score. The separate scores were averaged over the three sites into a staining intensity and percent positivity average score. A dual classification system was used for each specimen where the nuclear stain intensity is listed first, followed by the percent positivity of the cells stained listed second.

Three cases in the Good Outcome group did not have values for one of the sites. These were averaged over two sites only. This accounts for an average score of 3.5, whereas all others scores change by thirds.

TABLE 4
Site I

| | | | | | | | | | | |
|---|---|---|---|---|---|---|---|---|---|---|
| SPECIMEN | 1 | 2 | 3 | 4 | 5 | 6 | 7 | 8 | 9 | 10 |

Invasive Ductal Carcinoma

| | | | | | | | | | | |
|---|---|---|---|---|---|---|---|---|---|---|
| Epithelium | ⅔ | ½ | ⅔ | ⅔ | ⅔ | ⅔ | ⅔ | ⅔ | ⅔ | ⅔ |
| Ductal | ⅔ | ½ | 2/2 | 2/2 | ½ | ⅔ | ½ | ½ | ⅔ | ½ |
| Components | | | | | | | | | | |
| Inflammatory | ⅔ | 0- | ⅔ | ⅔ | ⅔ | ½ | ⅔ | ⅔ | ⅔ | ⅔ |
| Cells | | user | | | | | | | | |
| | | error | | | | | | | | |

Normal Breast

| | | | | | | | | | | |
|---|---|---|---|---|---|---|---|---|---|---|
| Epithelium | ⅔ | ¾ | ⅔ | ⅔ | ⅔ | ⅔ | ⅔ | 3/3 | 3/3 | 3/3 |
| Ductal | 3/3 | 3/3 | 3/3 | 3/3 | 3/3 | ⅔ | ¾ | ¾ | ⅔ | ¾ |
| Components | | | | | | | | | | |

TABLE 5
Site II

| | | | | | | | | | | |
|---|---|---|---|---|---|---|---|---|---|---|
| SPECIMEN | 11 | 12 | 13 | 14 | 15 | 16 | 17 | 18 | 19 | 20 |

Invasive Ductal Carcinoma

| | | | | | | | | | | |
|---|---|---|---|---|---|---|---|---|---|---|
| Epithelium | ⅔ | ½ | 2/2 | ⅔ | 2/2 | ⅔ | ⅔ | ⅔ | ⅔ | ⅔ |
| Ductal | 2/2 | ½ | ⅔ | 2/2 | ½ | ⅔ | ½ | ½ | ⅔ | ½ |
| Components | | | | | | | | | | |
| Inflammatory | ⅔ | 1/1 | 3/3 | ⅔ | ⅔ | ½ | ⅔ | ⅔ | ⅔ | ⅔ |
| Cells | | | | | | | | | | |

Normal Breast

| | | | | | | | | | | |
|---|---|---|---|---|---|---|---|---|---|---|
| Epithelium | 2/4 | 3/3 | ⅔ | ⅔ | ⅔ | ⅔ | ⅔ | ⅔ | 3/3 | 3/3 |
| Ductal | 3/3 | ¾ | 3/3 | ⅔ | 3/3 | 3/3 | ¾ | ¾ | ⅔ | 4/4 |
| Components | | | | | | | | | | |

TABLE 6
Site III

| | | | | | | | | | | |
|---|---|---|---|---|---|---|---|---|---|---|
| SPECIMEN | 1 | 2 | 3 | 4 | 5 | 6 | 7 | 8 | 9 | 10 |

Invasive Ductal Carcinoma

| | | | | | | | | | | |
|---|---|---|---|---|---|---|---|---|---|---|
| Epithelium | ⅔ | 2/2 | ⅔ | ⅔ | ½ | ⅔ | ⅔ | ½ | ⅔ | ⅔ |
| Ductal | ½ | 2/2 | 2/2 | ½ | 2/2 | ⅔ | ½ | 2/2 | ⅔ | ½ |
| Components | | | | | | | | | | |
| Inflammatory | 2/2 | ⅔ | ⅔ | 2/2 | ½ | ½ | ⅔ | ⅔ | ⅔ | 2/2 |
| Cells | | | | | | | | | | |

Normal Breast

| | | | | | | | | | | |
|---|---|---|---|---|---|---|---|---|---|---|
| Epithelium | 3/3 | 3/3 | ⅔ | ⅔ | ⅔ | 4/4 | ⅔ | ⅔ | ¾ | ¾ |
| Ductal | ⅔ | ¾ | ¾ | ⅔ | 3/3 | 3/3 | ¾ | ¾ | ⅔ | ¾ |
| Components | | | | | | | | | | |

### Example 6

### Discriminant Analysis (DA) and Logistic Regression (LR)

The statistical analysis involved in the present study included consideration of several factors. First the agreement between sites was examined. Simple correlations and kappa scores of agreement were used to examine the degree of agreement between sites. The analysis showed that agreement was not very satisfactory. Agreement between Positivity and Intensity within each site was fairly high (correlations in the 0.5 range), but agreement between sites was fairly low (correlations in the 0.2 to 0.3 range). Sites I and III agreed more with each other than with Site II. As a consequence, it was decided to use average Intensity and Positivity scores across three sites. For three cases data from one site was missing. For those the average was calculated from the remaining two sites. Data from at least two sites were always available. Average Scores increased the probability of correct classification considerably.

### Discriminant Analysis and Logistic Regression

Discriminant Analysis and Logistic Regression were both used to classify cases. Both Discriminant Analysis (DA) and Logistic Regression (LR) result in case scores that are turned into estimated probabilities of group membership. The two groups here are GOOD outcome and POOR outcome. The two methods yielded slightly different probabilities of group membership, but virtually the same classification of the 360 cases into POOR and GOOD. Other methods, with different assumptions, such as Cluster Analysis and Fuzzy Partitioning also yielded very similar results.

For discriminant analysis based on randomly split data, the data were randomly split into two sets each about half the original data set. DA and LR parameters were calculated based on one half of the data and used to predict the classification of the other half of the data. The agreement between split and full data analysis was very close.

### Probability Calculations

Calculations to determine the probability that FAS measurement scores correctly classified POOR and GOOD outcomes were performed. The available data set only contained cases that morphological methods already identified as GOOD. Therefore resulting probabilities are restricted to such cases. In this step, which is combined with Step 2 on statistical packages, the probabilities of group membership are used to predict (or classify) cases as GOOD or POOR resulting in predicted group membership for each case. Predicted group membership can be cross-classified with actual group membership in a 2×2 contingency table.

From the results, the following probabilities (“P”) of misclassification were estimated:

P[FAS predicts poor outcome|good outcome group]=23/180=0.128

P[FAS predicts good outcome|poor outcome group]=42/180=0.233

The backward conditional probabilities estimate the probability of an outcome given a certain FAS measurement score test result:

P[Good Outcome|FAS good outcome]=157/(157+42)=0.788

P[Poor Outcome|FAS poor outcome]=138/(138+23)=0.857

The sample contains exactly 180 cases with GOOD outcome and 180 cases with POOR outcome (50% GOOD, 50% POOR) while the actual population contained approximately 70% GOOD and 30% POOR outcomes. The population adjusted backward conditional probabilities were:

P[Good Outcome|FAS good outcome]=0.61/0.68=0.90

P[Poor Outcome|FAS poor outcome]=0.23/0.32=0.72

These results show that FAS measurement score agrees with morphological methods when they are correct. FAS predicted GOOD outcome for 87% of the GOOD outcome cases. Morphological methods predicted GOOD for 100% of the cases, for these are the only ones included in the study. The results also show that FAS measurement score disagrees with morphological methods when they are wrong. Consequently, FAS measurement score correctly classifies 76.7% of all the POOR outcome cases that morphological methods falsely called GOOD.

### Development of Scoring Cut-Offs Based on Classification Probabilities

Statistically, whenever the P[GOOD]>0.5, then a case is classified as GOOD. This criterion yields cut-off points based on Logistic Regression as shown in Table 7. The data are based on the scoring system outlined in Tables 2-3 and the data of Tables 4-6 and includes the dual scores for all of the epithelial, ductal and inflammatory cells for cancer patients and the epithelial and ductal components for normal tissues.

TABLE 7
Boundary Pairs for (Intensity, Positivity) readings of FAS expression
Classify as Good outcome IF

| Stain Intensity is at least | AND Positivity is at least |
|---|---|
| 2.45 | 3.69 |
| 2.69 | 3.23 |
| 3.10 | 3.00 |
| 3.23 | 2.55 |
| 3.69 | 2.45 |
| 3.98 | 2.00 |

DA and LR yield slight different cut-offs. DA is more willing to classify a case as GOOD than is LR.

Comparison with her-2/Neu and Estrogen Receptor Tests

The results from these two tests were cross-tabulated with actual group membership and also included as predictor terms in LR.

Neither of these tests reached the correct classification probabilities of the average FAS-score, nor were they helpful as additional terms in a LR model. HER-2/neu was not significant and ER actually counterproductive, reducing the correct classification probabilities noticeably.

### Discriminant Analysis of all Cases

Discriminant analysis is a multivariate statistical technique used to classify subjects into two or more non-overlapping populations. In traditional discriminant analysis one uses the known group membership of the subjects to derive a linear function of the score variables to optimize prediction of group membership. From the scores of the discriminant function one can derive a probability of group membership. Clustering and Logistic Regression are alternative methods that also calculate probabilities of group membership for each case. These differ from those obtained in discriminant analysis.

In the FAS study there were two populations, Poor (Outcome) and Good (Outcome). Accordingly, discriminant analysis calculates two probabilities.
1. P[Poor Outcome] is the estimated probability that a case belongs to the Poor Outcome group.
2. P[Good Outcome] is the estimated probability that a case belongs to the Good Outcome group.

### Hence, P[Poor Outcome]+P[Good Outcome]=1.

If P[Poor Outcome]>P[Good Outcome], i.e., if P[Poor Outcome]>0.5, then a case is predicted to belong to the Poor Outcome group and vice versa. A variable “Pred group” is the prediction of discriminant analysis as to group membership.

The formulas for all 360 cases are reminiscent of a quadratic regression equation. Essentially one calculates for each case a group score for the Good and the Poor group. These formulas for the group scores are given as Dist[good] and Dist[poor] respectively.

Dist[good]=60.7262835−14.779283*avgIntensity+4.97420821*avgIntensitŷ2−22.42291*avgPositivity+5.94409261avgPositivitŷ2−5.2174324*avgIntensity*avgPositivity

Dist[poor]=37.8423625−10.183222*avgIntensity+4.97420821*avgIntensitŷ2−19.098415*avgPositivity+5.94409261avgPositivitŷ2−5.2174324*avgIntensity*avgPositivity

The scores are used to calculate the probability of group membership:

P[good]=exp{−0.5*Dist[good]}/P[Sum]

P[poor]=exp{−0.5*Dist[poor]}/P[Sum]

Where

P[Sum]=exp{−0.5*Dist[good]}±exp{−0.5*Dist[poor]}

Table 8 shows the results for subjects 1 and 2 of the Good Outcome group and subjects 181 and 182 of the Poor Outcome group.

TABLE 8
Four Sample results of Discriminant Analysis

| | | avg | avg | Prob | Prob | |
|---|---|---|---|---|---|---|
| | | (staining | (percent | [Poor | [Good | Pred |
| case | group | intensity) | positivity) | Outcome] | Outcome] | group |
| 1 | good | 3.223 | 3.223 | 0.145 | 0.828 | Good |
| | | | | | | Outcome |
| 2 | good | 3.223 | 3.223 | 0.145 | 0.843 | Good |
| | | | | | | Outcome |
| 181 | poor | 3.223 | 2.669 | 0.242 | 0.669 | Good |
| | | | | | | Outcome |
| 182 | poor | 2.223 | 3.100 | 0.728 | 0.261 | Poor |
| | | | | | | Outcome |

As can be seen, for cases 1, 2, and 182 the actual group membership variable “group” agrees with “Pred group” and so they are correctly classified as GOOD, GOOD and POOR Outcomes respectively. Case 181 shows a difference between “group” and “Pred group” and represents a misclassification.

A cross-classification of actual “group” membership versus “Pred group” results Table 9. The table shows that discriminant analysis would have classified 138 of the 180 Poor Outcome cases correctly, but would have called 42 Good Outcome. This is a false negative rate of 23.3%. Of the Good Outcome cases, discriminant analysis would have predicted 157 correctly, with 23 false positives—a 12.8% error rate. The overall error rate is 65 out of 360 or 18.1%.

TABLE 9
Cross-classification of predicted group
membership with actual group membership of 180
GOOD and 180 POOR cases based on Discriminant Analysis

| | | group | | |
|---|---|---|---|---|
| Pred Group | | Poor | Good | Total |
| | Poor Outcome | 138 | 23 | 161 |
| | Good Outcome | 42 | 157 | 199 |
| | Total | 180 | 180 | 360 |

The Kappa coefficient of intrarater reliability is 0.599639 with a Std Err of 0.040286, which is fairly high and statistically significant.

From this Table one can estimate the following probabilities of misclassification:

P[FAS predict poor outcome|good outcome group]=23/180=0.128

P[FAS predict good outcome|poor outcome group]=42/180=0.233

The complete estimated classification probabilities are in Table 10:

TABLE 10
Estimated prediction probabilities

| | | group | | |
|---|---|---|---|---|
| Pred group | Counts | Poor | Good | Total |
| | Poor Outcome | .767 | .128 | .44 |
| | Good Outcome | .233 | .872 | .55 |
| | Prob in Sample | .5 | .5 | 1 |
| | EST. Prob in Pop | .3 | .7 | 1 |

The backward conditional probabilities estimate the probability of an outcome given a certain FAS test result:

P[Good Outcome|FAS good outcome]=157/(157+42)=0.788

P[Poor Outcome|FAS poor outcome]=138/(138+23)=0.857

The sample contains exactly 180 cases with GOOD outcome and 180 cases with POOR outcome (50% GOOD, 50% POOR. These probabilities are based on the assumption that the sample proportion of GOOD and POOR outcomes reflects the population proportions.

The sample was taken from a population that morphological methods classified entirely as GOOD. This population contained approximately 30% poor outcomes. So to estimate the success of FAS classification one must adjust the classification probabilities from the sample probabilities to those in the population (70% GOOD, 30% Poor) by multiplying the estimated probabilities accordingly. For example, 0.767*0.3=0.23=P[FAS classifies a cases as poor and that case has indeed poor outcome in the population]. This yields the following adjusted table of probabilities:

TABLE 11
Estimated prediction probabilities adjusted for 70/30
representation of GOOD/POOR in available samples

| | | group | | |
|---|---|---|---|---|
| Pred group | Counts | Poor | Good | Total |
| | Poor Outcome | .23 | .09 | .32 |
| | Good Outcome | .070 | .61 | .68 |
| | EST. Prob in Pop | .30 | .70 | 1 |

The adjusted backward conditional probabilities are:

P[Good Outcome|FAS good outcome]=0.61/0.68=0.90

P[Poor Outcome|FAS poor outcome]=0.23/0.32=0.72

These probabilities state that in a population containing only cases that had been identified as GOOD by morphological methods, GOOD results on a FAS test carries with it a 0.90 probability that the case is indeed GOOD. Likewise, a POOR result on a FAS test carries with it a 0.72 probability that the case is indeed POOR.

Using the discrimination formulas the following probabilities of group membership can be arrived at assuming average scores. The predicted Good Outcome Group consists of scores (Intensity, Positivity)={(3,3), (3,4), (4,2), (4,3), (4,4)}. These four groups have a P[good]>0.5.

### Logistic Regression

Logistic Regression yields virtually the same classification as did discriminant analysis. Only one case, with site scores of (4/3, 2/1, 4/3) and average scores of (3.33, 2.33) would have been classified as Good with discriminant analysis (P[poor]=0.475), and as Poor with logistic regression (P[poor]=0.509.

### Example 7

### Analysis of Other Variables

### Age as Additional Covariate

As additional variable the age of each case (patient) was entered into the Logistic Regression model. It was determined that age did not produce a significant contribution (p=0.39).

### HER-2/Neu

A cross-classification of actual “group” membership versus HER results was performed. The result showed that HER-2/Neu would have classified 85 of the 180 Good Outcome cases as negative, and would have called 95 positive. This is a false positive rate of 52.8% (compared to 23.3% for average FAS). Of the Poor Outcome cases, HER-2/Neu would have predicted 109 (60.5%) correctly, with 71 false negatives—a 39.4% error rate compared to 12.3% for the FAS average. The overall error rate is (95+71) out of 360 or 46.1%. This simple classification shows that HER-2/Neu is considerably inferior to the FAS measurement scores in prediction outcome. In fact, there is hardly a noticeable reduction in error, although the results are significant.

### ER-Estrogen Receptor

A cross-classification of actual “group” membership versus ER was performed. The data showed that ER would have classified 90 of the 180 Good Outcome cases as negative, and would have called 90 positive. This is a false positive rate of 50% (compared to 23.3% for average FAS). Of the Poor Outcome cases, ER would have predicted 128 (71.1%) correctly, with 52 false negatives—a 28.9% error rate compared to 12.3% for the FAS average. The overall error rate was (90+52) out of 360 or 39.4% compared with 18.1% of the FAS average. This classification showed that ER is better than HER-2Neu in classifying outcome correctly, but still considerably inferior to the FAS average scores. In fact, there is small reduction in error, and the results are significant. Practically they appear of very limited value.

In summary, FAS appears to have considerable diagnostic value above and beyond morphological methods. FAS measurement found over 85% of the POOR cases morphological methods missed and was in agreement 90% when morphological methods correctly classified as good. HER-2/neu and ER proved to inferior to FAS on this data set. The age of patient was not a significant predictor. Statistical methods based on a variety of assumptions seem to produce similar results.

### Example 8

### FAS Standard ELISA Colorimetric or Chemiluminescence Protocol

The following is an outline of the procedures for the development, optimization and documentation of novel antibodies using serial dilution and automated Immunohistochemistry (IHC) staining.

### DEFINITION

PBS: Phosphate Buffered Saline is a buffer solution commonly used in biological research. It is a water-based salt solution containing sodium chloride, sodium phosphate, and (in some formulations) potassium chloride and potassium phosphate. The buffer helps to maintain a constant pH. The osmolarity and ion concentrations of the solution usually match those of the human body (isotonic).

### Equipment

Ventana Benchmark XT Staining System or equivalent

### Materials

PPE: personal protective equipment

Microcentrifugr tubes

15 mL conical tubes

PBS or diluent

Pipette

Pipette tips

FAS Antibodies

### Procedure

1. Coat wells on a 96 well microplate with 100 μl/well of coating antibody diluted in PBS.
2. Incubate plate overnight at 4° C., covered with plate sealer.
3. Wash plate 5 times with 300 ul of PBS-T (0.05% Tween 20) on Wellwash Versa Plate washer (Thermo).
4. Block plates with 300 μl/well of ELISA Blocker Blocking Solution (Thermo) for 2 hours at 23° C. with shaking at 100 rpm in Incubating Microplate Shaker (VWR) covered with plate sealer.
5. Wash plates 5 times with PBS-T 300 μl/well on plate washer.
   After each washing step, tap plate onto kimwipes on bench to remove any excess liquid.
6. Load 100 μl of standards or samples freshly diluted in PBS-T 23° C. on plate shaker with 100 rpm agitation for 2 hours, covered with plate sealer.
   Prepare protein standards ahead of time on ice.
7. Freshly prepare a 7-point dilution. e.g. from 400 ng/ml to 1 ng/ml in 1% BSA/PBS-T.
8. Wash plate 5 times with PBS-T 300 ul/well on plate washer.
9. Incubate 100 l/well of biotinylated detection antibody diluted in PBS to appropriate concentration for 2 hours at 23° C. on plate shaker with 100 rpm agitation, covered with plate sealer.
10. Wash plate 5 times with PBS-T 300 ul/well on plate washer.
11. Incubate 100 l/well of streptavidin-HRP (R&D Inc.), 1:200 dilution in PBS at 23° C. on plate shaker with 100 rpm agitation for 20 minutes, covered with plate sealer.
12. Wash plate 5 times with PBS-T 300 ul/well on plate washer.
   For colorimetric measurement, add 100 ul of Tetramethylbenzidine substrate solution (Thermo) to each well.
13. Incubate for 20 minutes at room temperature in the dark.
14. Add 50 ul of acidic Stop solution (Thermo) to stop color development.
15. Determine the optical density of samples with BioTek FL800x plate reader at 450 nm,
   For chemiluminescence measurement, amplify signal by adding 100 ul/well Gloset Substrate (R&D Inc.), for 5 to 15 minutes at room temperature inside a BioTek FL800x plate reader. Prepare Substrate ahead of time. Freshly prepare by mixing Reagent A: Reagent B 1:2.
16. Set signal measured on BioTek FL800x fluorometer at 0.5 second read time with sensitivity, auto adjusted to highest point on standard curve, and set to a reading of 400,000.

It should be noted that ELISA Sandwich assays useful in the present invention include those as described in PCT Publication PCT/US2010/046773 published Mar. 17, 2011, the contents of which are incorporated here by reference in its entirety.

### Example 9

### Use of Commercial FAS Antibodies

IHC and ELISA assays may be performed using a commercial anti-FAS antibody. For IHC the antibodies used are human anti-FASN Antibody, Affinity Purified (Catalog No. A301-324A) from Bethyl Laboratories (Montgomery, Tex.). For ELISA studies, the antibodies used are the Fatty Acid Synthase Antibody Pair (Catalog No. H00002194-AP11) from Novus Biologicals (Littleton, Colo.). The pair contains a Capture antibody which is rabbit affinity purified polyclonal anti-FASN (100 ug) and a Detection antibody which is mouse monoclonal anti-FASN, IgG1 Kappa (20 ug).

### Example 10

### Preparation of Anti-FAS Monoclonal Antibodies

Anti-FAS antibodies and an immunohistochemical ELISA assay employing the antibodies are disclosed in PCT Publication PCT/US2010/030545 published Oct. 14, 2010, and PCT/US2010/046773 published Mar. 17, 2011, respectively. The contents of each are incorporated here by reference in their entirety.

Briefly, four murine monoclonal antibodies were prepared by immunizing SCID mice with synthetic FAS peptides, and establishing hybridomas according to the general procedure described by Iyer et al., Ind. J. Med. Res., 123:651-564 (2006). Each mouse was immunized with one peptide of SEQ ID NOs 1-5.

FAS Peptides:

SEQ ID NO. 1
VAQGQWEPSGXAP
SEQ ID NO. 2
PSGPAPTNXGALE
SEQ ID NO. 3
TLEQQHXVAQGQW
SEQ ID NO. 4
EVDPGSAELQKVLQGD
SEQ ID NO. 5
ELSSKADEASELAC

Humanized monoclonal antibodies were prepared as described by Carter et al., Proc. Natl. Acad. Sci. USA, 89:4285-89 (1992) from monoclonal antibodies derived from hybridomas A, B, D and E. The humanized monoclonal antibodies (MAbs) are referred to hereinafter as FAS 1, FAS 2, FAS 4 (ATCC Deposit No: PTA-10811) and FAS 5 (ATCC Deposit No: ______), respectively.

### Example 11

### FAS Detection in Nipple Aspirate Fluid

Ductal fluid was collected by nipple aspiration using a modified breast pump used to express milk from women with breast cancer. The modified breast pump consisted of a plastic cup connected to a section of polymer tubing which was then attached to a standard syringe in order to create a gentle vacuum. Before aspiration, the nipple was cleansed in order to remove keratin plugs, and then with an alcohol pad. The breast was allowed to dry before a warm moist cloth was placed on the breast for about 1-2 minutes. After the damp cloth was removed from the breast, the breast was gently massaged from the chest wall toward the nipple from about a minute. The suction cup was then placed over the nipple and the plunger of the syringe was withdrawn to the 5- to 10-ml level until ductal fluid was visualized. The fluid droplets were collected and sample volumes of the nipple aspirate fluid were recorded.

After collection, the nipple aspirate fluid samples were rinsed into centrifuge tubes containing 500 μL of sterile PBS supplemented with protease inhibitors 4-[2-aminoethyl]-benzenesulfonylfluoride-HCl (0.2 mmol/L), leupeptin (50 g/mL), aprotinin (2 g/mL), and DTT (0.5 mmol/L). The samples were then centrifuged at 1500 rpm for 10 minutes in order to remove insoluble materials. The supernatant was collected in 50 μL aliquots which were processed to determine the intensity of the nuclear stain. The intensity level for each sample was classified using the classification system listed in Table 2 in order to determine the expression level of FAS contained in the samples.

35 nipple aspirate samples were collected and analyzed, as described above, in order to determine the levels of FAS expression in women with breast cancer. The samples collected from women over 50 years of age who had infiltrating ductal carcinoma were found to have a decreased chance of survival after 5 years when the expression of FAS in their nipple aspirate fluid was greater than 1. Table 12 is a listing of the 35 nipple aspirate samples collected and analyzed.

TABLE 12
FAS Expression Levels from Nipple Aspirate Fluid Samples

| | | | | | Chemo- | Radiation | 5 year |
|---|---|---|---|---|---|---|---|
| Age | Histology | Stage | FAS | Surgery | therapy | Therapy | Survival |
| 39 | Lobular | 2 | 1 | Mastec- | CMF | Radiation | Yes |
| | carcinoma | | | tomy | | | |
| 39 | Lobular | 2 | 2 | Mastec- | CMF | Radiation | Yes |
| | carcinoma | | | tomy | | | |
| 39 | Matched | | 2 | Mastec- | | Radiation | Yes |
| | benign | | | tomy | | | |
| | specimen | | | | | | |
| 40 | Infiltrating | 2 | 2 | Biopsy | | Radiation | Yes |
| | ductal | | | | | | |
| | carcinoma | | | | | | |
| 41 | Tubular | 2 | 1 | Biopsy | | Radiation | Yes |
| | carcinoma | | | | | | |
| 41 | Tubular | 2 | 1 | Biopsy | | Radiation | Yes |
| | carcinoma | | | | | | |
| 41 | Matched | | 1 | Biopsy | | Radiation | Yes |
| | benign | | | | | | |
| | specimen | | | | | | |
| 46 | Infiltrating | 3 | 3 | Mastec- | 5-FU | Radiation | Yes |
| | ductal | | | tomy | | | |
| | carcinoma | | | | | | |
| 46 | Infiltrating | 3 | 3 | Mastec- | 5-FU | Radiation | Yes |
| | ductal | | | tomy | | | |
| | carcinoma | | | | | | |
| 46 | Matched | | 3 | Mastec- | | Radiation | Yes |
| | benign | | | tomy | | | |
| | specimen | | | | | | |
| 52 | Infiltrating | 1 | 2 | Biopsy | | Radiation | No |
| | ductal | | | | | | |
| | carcinoma | | | | | | |
| 52 | Infiltrating | 1 | 2 | Biopsy | | Radiation | No |
| | ductal | | | | | | |
| | carcinoma | | | | | | |
| 52 | Matched | | 2 | Biopsy | | Radiation | Yes |
| | benign | | | | | | |
| | specimen | | | | | | |
| 55 | Infiltrating | 1 | 1 | Biopsy | | Radiation | Yes |
| | ductal | | | | | | |
| | carcinoma | | | | | | |
| 55 | Matched | | 1 | Biopsy | | Radiation | Yes |
| | benign | | | | | | |
| | specimen | | | | | | |
| 61 | Infiltrating | 2 | 1 | Mastec- | Adria/ | Radiation | Yes |
| | ductal | | | tomy | CMF | | |
| | carcinoma | | | | | | |
| 61 | Infiltrating | 2 | 1 | Mastec- | Adria/ | Radiation | Yes |
| | ductal | | | tomy | CMF | | |
| | carcinoma | | | | | | |
| 61 | Scirrhous | 1 | 1 | Biopsy | | Radiation | Yes |
| | carcinoma | | | | | | |
| 65 | Lobular | 2 | 2 | Biopsy | | Radiation | Yes |
| | carcinoma | | | | | | |
| 65 | Lobular | 2 | 2 | Biopsy | | Radiation | Yes |
| | carcinoma | | | | | | |
| 65 | Lobular | 2 | 2 | Biopsy | | Radiation | Yes |
| | carcinoma | | | | | | |
| 65 | Medullary | 2 | 3 | Mastec- | CMF | Radiation | Yes |
| | carcinoma | | | tomy | | | |
| 65 | Medullary | 2 | 3 | Mastec- | CMF | Radiation | Yes |
| | carcinoma | | | tomy | | | |
| 69 | Infiltrating | 3 | 4 | Mastec- | Adria/ | Radiation | No |
| | ductal | | | tomy | CMF | | |
| | carcinoma | | | | | | |
| 69 | Infiltrating | 3 | 4 | Mastec- | Adria/ | Radiation | No |
| | ductal | | | tomy | CMF | | |
| | carcinoma | | | | | | |
| 86 | Infiltrating | 3 | 4 | Biopsy | | Radiation | Yes |
| | ductal | | | | | | |
| | carcinoma | | | | | | |
| 86 | Infiltrating | 3 | 4 | Biopsy | | Radiation | Yes |
| | ductal | | | | | | |
| | carcinoma | | | | | | |
| Unk | Infiltrating | 2 | 2 | Mastec- | CMF | Radiation | No |
| | ductal | | | tomy | | | |
| | carcinoma | | | | | | |
| Unk | Infiltrating | 2 | 2 | Mastec- | CMF | Radiation | No |
| | ductal | | | tomy | | | |
| | carcinoma | | | | | | |
| Unk | Infiltrating | 2 | 1 | Mastec- | CMF | Radiation | Yes |
| | ductal | | | tomy | | | |
| | carcinoma | | | | | | |
| Unk | Infiltrating | 2 | 1 | Mastec- | CMF | Radiation | Yes |
| | ductal | | | tomy | | | |
| | carcinoma | | | | | | |
| Unk | Infiltrating | 1 | 1 | Mastec- | | Radiation | Yes |
| | ductal | | | tomy | | | |
| | carcinoma | | | | | | |
| Unk | Infiltrating | 1 | 1 | Mastec- | | Radiation | Yes |
| | ductal | | | tomy | | | |
| | carcinoma | | | | | | |
| Unk | Scirrhous | 1 | 1 | Mastec- | | Radiation | Yes |
| | carcinoma | | | tomy | | | |
| Unk | Scirrhous | 1 | 1 | Mastec- | | Radiation | Yes |
| | carcinoma | | | tomy | | | |

For women diagnosed with infiltrating ductal carcinoma the data show that irrespective of cancer stage, type of surgery or diameter of tissue sample, where FAS levels in nipple aspirates are 2 or greater and the resected margin is positive, there is a less than 5 year survival rate. The data are shown in Table 13.

TABLE 13
Five year survival when FAS greater than 2 and Positive Resected Margin

| | | | Resected | | | Radiation | | 5 year |
|---|---|---|---|---|---|---|---|---|
| Age | Diameter | Surgery | Margin | Stage | FAS | Therapy | Chemotherapy | Survival |
| 52 | 2.0 cm | Biopsy | Positive | 1 | 2 | Radiation | | No |
| 52 | 2.0 cm | Biopsy | Positive | 1 | 2 | Radiation | | No |
| Unk | | Mastectomy | Positive | 2 | 2 | Radiation | CMF | No |
| Unk | | Mastectomy | Positive | 2 | 2 | Radiation | CMF | No |
| 69 | 6.0 cm | Mastectomy | Positive | 3 | 4 | Radiation | Adria/CMF | No |
| 69 | 6.0 cm | Mastectomy | Positive | 3 | 4 | Radiation | Adria/CMF | No |

1-20. (canceled)

21. A method for predicting a clinical outcome of a patient diagnosed with lymph node negative breast cancer, the method comprising:
(a) obtaining a tissue sample from the patient by excision, aspiration or biopsy,
(b) contacting the sample with a fatty acid synthase (FAS)-specific antibody comprising an isolated monoclonal antibody that specifically binds to an amino acid selected from the group consisting of SEQ ID NOs. 4 and 5,
(c) measuring the binding properties of the antibodies to FAS protein in the sample,
(d) determining the expression level of FAS protein in the sample according to the binding properties in step (c) and comparing such expression level with that in a control sample from a normal subject, and
(e) correlating the expression level of FAS protein with one or more clinical management parameters of the patient,
wherein an elevated expression level of FAS protein in the sample indicates a good clinical outcome of the patient.

22. The method of claim 21, wherein the sample is selected from the group consisting of epithelial cells or tissue, ductal components and inflammatory cells.

23. The method of claim 21, wherein the binding properties of the antibodies to FAS are determined by an immunoassay.

24. The method of claim 23, wherein said FAS-specific antibody further comprises a detectable label.

25. The method of claim 23, wherein the immunoassay is selected from the group consisting of an immunohistochemical assay and an enzyme-linked immunosorbent assay (ELISA).

26. The method of claim 21 further comprising measuring one or more additional biomarkers for breast cancer in the sample.

27. The method of claim 21 further comprising combining the morphological features of the breast of the patient, wherein the morphological features include primary tumor type, status of the reaction margin, evidence of skin, nipple or lymphatic vessel invasion and determination of the extent of an in-situ component.

28. The method of claim 21, wherein the clinical management parameters include survival in years, disease related death, early or late recurrence, degree of regression, metastasis, responsiveness to treatment and or effectiveness of treatment.

29. The method of claim 21, wherein the good clinic outcome is five years free of breast cancer in the patient.


SEQUENCE LISTING (6 sequences)

SEQ ID NO: 1 - protein, 13 aa, Human
  misc_feature (11)..(11): Xaa can be any naturally occurring amino acid
VAQGQWEPSGXAP

SEQ ID NO: 2 - protein, 13 aa, Human
  misc_feature (9)..(9): Xaa can be any naturally occurring amino acid
PSGPAPTNXGALE

SEQ ID NO: 3 - protein, 13 aa, Human
  misc_feature (7)..(7): Xaa can be any naturally occurring amino acid
TLEQQHXVAQGQW

SEQ ID NO: 4 - protein, 17 aa, Human
EVADPGSAELQKVLQGD

SEQ ID NO: 5 - protein, 14 aa, Human
ELSSKADEASELAC

SEQ ID NO: 6 - DNA, 8481 bp, Homo sapiens
  CDS (118)..(7653)
gagagacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagatgtaggtgttaggcatgtcccacccacccgccgcctcccacggcacctcggggacaccagagctgccgacttggagactcctggtctgtgaagagccggtggtgcccgtgcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagcccgctgagtggggggtcgtgggccagtccccgaggagctgggcccctgcacaggcacacagggcccggccacacccagcggccccccgcacagccacccgtggggtgctgcccttatgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctccaaaaaaaaaaaaaaaaaaaaaaaa